(19) United States
(12) Patent Application Publication
(10) Pub. No.: US 2010/0256230 A1
(43) Pub. Date: Oct. 7, 2010

(54) 1-BUTANE ACID DERIVATIVES, PHARMACEUTICAL COMPOSITIONS CONTAINING SAID DERIVATIVES AND THE USE THEREOF

(75) Inventors: Erich Eigenbrodt, Linden (DE); Stefan Mullner, Langenfeld (DE); Sybille Mazurek, Linden (DE); Hugo Fasold, Frankfurt (DE)

Correspondence Address: MAYER & WILLIAMS PC, 251 NORTH AVENUE WEST, 2ND FLOOR, WESTFIELD, NJ, 07090, US

(73) Assignee: SCHEBO BIOTECH AG, Giessen (DE)

(21) Appl. No.: 12/573,816

(22) Filed: Oct. 5, 2009

Related U.S. Application Data
(63) Continuation of application No. 10/471,703, filed on Aug. 9, 2004, now abandoned.

(30) Foreign Application Priority Data
Mar. 13, 2001 (DE) 101 12 924.6

(51) Int. Cl. A61K 31/275; C07C 255/20; A61P 35/00; A61P 29/00; A61P 19/00; A61P 7/08; A61P 1/02; A61P 43/00

(52) U.S. Cl. 514/527; 558/441

(57) ABSTRACT

The invention relates to new butane acid derivatives comprising a cyanide group, pharmaceutical compositions containing said butane acid derivatives and the use of said butane acid derivatives in the production of pharmaceutical compositions for treating various illnesses.

### STATEMENT OF RELATED APPLICATIONS

This is a continuation of U.S. patent application Ser. No. 10/471,703, filed Aug. 9, 2004, entitled “1-Butane Acid Derivatives, Pharmaceutical Compositions Containing Said Derivatives and The Use Thereof,” which is a U.S.C. 371 National Stage Application of International Patent Application Serial No. PCT/DE02/00922, filed Mar. 12, 2002, which claims the benefit of German Patent Application Serial No. 101 12 924.6, filed Mar. 13, 2001. Each of the prior applications is incorporated herein by reference in its entirety.

### FIELD OF THE INVENTION

The invention relates to butane acid derivatives, pharmaceutical compositions containing said derivatives and the use of said derivatives in the production of pharmaceutical compositions for treating various illnesses.

### BACKGROUND OF THE INVENTION

Cancer today is one of the most frequent causes of death, and the number of cancer cases in the industrialized countries continuously grows. This is mainly because malignant tumors are a disease of higher age, and due to a successful control of infection diseases, more people will reach this age. In spite of all progress in the diagnostic and therapeutic field, the healing chances for the most frequent inner cancer types are seldom higher than 20%. A cancerous tumor nowadays can be destroyed or inhibited in its growth. A re-conversion of a tumor cell into a normal cell is however not yet possible. The most important therapeutic measures, the operation and the irradiation, remove cancer cells from the organism. The presently used chemotherapeutic agents of cancer, the cytostatics, also lead to a destruction or damaging of tumor cells only. In most cases, the effect is of such low specificity that simultaneous heavy damages to healthy cells will occur.

In general, tumor cells have a metabolism differing from healthy cells, in particular glycolysis. Thus, a change of the isoenzyme system involved in the glycolysis and a change of the transport of NADH is typical for tumor cells. Among other effects, the activity of the enzymes of the glycolysis is increased. This permits high reaction rates under the aerobic conditions typical for tumor cells. For details, reference is made to E. Eigenbrodt et al., Biochemical and Molecular Aspects of Selected Cancers, Vol. 2, p. 311 ff, 1994.

### PRIOR ART

From the document E. Eigenbrodt et al., Biochemical and Molecular Aspects of Selected Cancers, Vol. 2, p. 311 ff, 1994 it is known in the art to use glucose analogs for inhibiting the glycolysis. Other approaches known are the use of inhibitors of glycolytic isoenzymes, for instance by suitable complex formation or inhibition of complex formation. As a result, tumor cells are, so to speak, “starved out”. It is a problem, however, with the above compounds that many of them are genotoxic and/or not sufficiently specific for tumor cells.

In conjunction with a new active ingredient against inflammatory illnesses it is known from the document U. Mangold et al., Eur. J. Biochem., 266:1-9, 1999, that these active ingredients, namely leflunomide derivatives, also affect the glycolysis.

### SUMMARY OF THE INVENTION

The invention is based on the technical object to provide active ingredients, which are capable of inhibiting the proliferation of cancer cells and thus the growth of neoplastic tumors. It is an object of the invention that these active ingredients also inhibit defensive over-reactions of the body, such as septic shock, autoimmune diseases, transplant rejections as well as acute and chronic inflammatory diseases, and simultaneously with little or no cytotoxicity with regard to normal cells of the blood, of the immune system and the tissue cells.

### BRIEF DESCRIPTION OF THE DRAWINGS

FIG. 1 is a graphical representation that illustrates a dose-dependent inhibition of tumor cell growth upon treatment of Novikoff-Zellen hepatoma cells with carbomethoxypropionyl cyanide (CMPC). The cell density was measured after four days of cultivation.

FIG. 2 is a graphical representation that illustrates a dose-dependent inhibition of tumor cell growth upon treatment of Novikoff-Zellen hepatoma cells with L-glycoserine. The cell density was measured after 4 days of cultivation.

FIG. 3 is a graphical representation that illustrates the inhibition of glycolysis of Novikoff-Zellen hepatoma cells by CMPC as measured by lactate produced for glucose consumed.

### DETAILED DESCRIPTION OF THE INVENTION

For achieving said technical object, the invention teaches a compound according to formula I

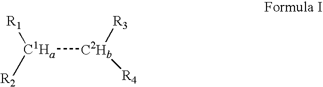

wherein a and b are identical or different and are 0 or 1,

wherein R1=—H, C1-C18 alkyl, cycloalkyl or aryl,

wherein R2=—OX1, —SX1, —COO⁻, —(CH₂)ₙ—COOX1 or —COOX1 with X1=—H, C1-C18 alkyl, cycloalkyl or aryl, and with n=1-8,

wherein R3=—CN, —COO⁻, —COOX2, —CO—X2, —CO—NHX2 with X2=—H, C1-C18 alkyl, cycloalkyl or aryl,

wherein R4=═O, —NHY or —CO—NHZ with Y=H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or C1-C8 alkyl, cycloalkyl or aryl substituted phenyl or with -Hal and/or —O-Hal and/or C1-C8 alkyl, cycloalkyl or aryl substituted naphthyl (-Hal=—F, —Cl, or —Br),

wherein a and b correspond to the number of remaining carbon valences at C¹ and C²,

wherein via R3 a ring connection to C¹ under elimination of X1 in R2 and X2 in R3 may be provided,

or of a physiologically well tolerated salt of such a compound

for the production of a pharmaceutical composition for the treatment and/or prophylaxis of illnesses from the group consisting of neoplastic tumors, inflammatory diseases, autoimmune diseases, degenerative joint diseases, rheumatic diseases with cartilage breakdown, chronic polyarthritis, joint trauma, immobilization-caused cartilage loss, septic shock, diseases with disturbed leukocyte adhesion, diseases by increased TNFalpha concentrations, cachexia, Crohn's disease, rejection reactions after transplantations.

Some substances covered by the above definitions are known per se and from other connections. Other substances covered by the above definitions are however novel. Therefore the invention further teaches compounds according to formula I

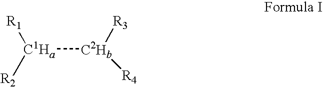

wherein a and b are identical or different and are 0 or 1,

wherein R1=—H, C1-C18 alkyl, cycloalkyl or aryl,

wherein R2=—OX1, —SX1, —COO⁻, —(CH₂)ₙ—COOX1 or —COOX1 with X1=—H, C1-C18 alkyl, cycloalkyl or aryl, and with n=1-8,

wherein R3=—CN,

wherein R4=═O, —NHY or —CO—NHZ with Y=H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or C1-C8 alkyl, cycloalkyl or aryl substituted phenyl or with -Hal and/or —O-Hal and/or C1-C8 alkyl, cycloalkyl or aryl substituted naphthyl (-Hal=—F, —Cl, or —Br),

wherein a and b correspond to the number of remaining carbon valences at C¹ and C², or a physiologically well tolerated salt of such a compound.

Finally, the invention teaches a pharmaceutical composition containing a compound according to formula I

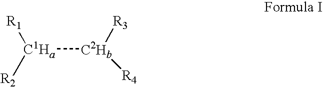

wherein a and b are identical or different and are 0 or 1,

wherein R1=—H, C1-C18 alkyl, cycloalkyl or aryl,

wherein R2=—OX1, —SX1, —COO⁻, —(CH₂)ₙ—COOX1 or —COOX1 with X1=—H, C1-C18 alkyl, cycloalkyl or aryl, and with n=1-8,

wherein R3=—CN, —COO⁻, —COOX2, —CO—X2, —CO—NHX2 with X2=—H, C1-C18 alkyl, cycloalkyl or aryl,

wherein R4=═O, —NHY or —CO—NHZ with Y=H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or C1-C8 alkyl, cycloalkyl or aryl substituted phenyl or with -Hal and/or —O-Hal and/or C1-C8 alkyl, cycloalkyl or aryl substituted naphthyl (-Hal=—F, —Cl, or —Br),

wherein a and b correspond to the number of remaining carbon valences at C¹ and C², wherein via R3 a ring connection to C¹ under elimination of X1 in R2 and X2 in R3 may be provided,

or a physiologically well tolerated salt of such a compound, and

at least one physiologically well tolerated auxiliary and/or carrier substance.

It is understood that for compounds according to formula I there may possibly exist stereoisomers, all of which are subject matter of the invention. The term alkyl comprises linear and branched alkyl groups. The term cycloalkyl also comprises cycloalkyl groups with linear or branched alkyl substituents. The term aryl also comprises aralkyl groups, wherein alkyl substituents may be alkyl or cycloalkyl.

Surprisingly it has been found that such 1-butane acid derivatives having the general formula (I) are capable of inhibiting in vitro, the proliferation of cancer cells when administered in therapeutically relevant concentrations in a dose-dependent manner. In the dose range investigated, there could not be found a cytotoxic effect. Due to their pharmacological properties, the compounds according to the invention are also excellently suitable for the treatment and prophylaxis of the illnesses named above.

For the invention, various non-limiting embodiments are possible. For instance, a pharmaceutical composition according to the invention may contain several different compounds covered by the above definitions. Further, a pharmaceutical composition according to the invention may in addition contain an active ingredient different from the compound of formula I. Then it is a combination preparation. Therein, the different used active ingredients may be prepared in one single dosage form, i.e. the active ingredients are mixed in the dosage form. It is however also possible to prepare the various active ingredients in spatially separated dosage forms of identical or different type.

It is preferred if the compounds according to the invention comprise the following groups:

R1=—H, methyl or ethyl,
R2=—OH, —COOH, —COO⁻, or —COOX with X=methyl or ethyl,
R4=═O, —NHY with Y=H or —COR (R=methyl, ethyl or —NHA with A=H, methyl or ethyl) or —CO—NHZ with Z=—F, —Br, —O—Cl, and/or —O—Br substituted phenyl.

Particularly suitable examples of compounds covered by formula I are described in the following.

Compound 1: R1=methyl, R2=—OH, R3=—CN, R4=—NH₂, a=b=0.

Compound 2: R1=methyl, R2=—OH, R3=—COOH, R4=—NH₂, a=b=0.

Compound 3: R1=methyl, R2=—OH, R3=—CN, R4=—NHY, a=b=0.

Compounds 4-6: R1=methyl, R2=—OH, R3=—CN, R4=—CO—NH—C₆H₄F (e.g. meta), —CO—NH—C₆H₃Br₂ (e.g. ortho, meta) or —C₆H₄OCl (e.g. para), a=b=0.

Compound 7: R1=methyl, R2=—OH, R3=—CN, R4=—CO—NH—Z, a=b=0.

Compound 8: R1=methyl, R2=—OH, R3=—CN, R4=—NH₂, a=b=0.

Compound 9: R1=—H, R2=—COO-methyl, R3=—CN, R4=═O, a=1, b=0.

Compound 10: R1=—H, R2=—COO⁻, R3=—COOH, R4=═O, a=1, b=0.

Compound 11: R1=—H, R2=—COO⁻, R3=—COOH, R4=—NH—CO—NH₂, a=b=1.

Compound 12: R1=—H, R2=—COO⁻, R3=—COOH, R4=NH₂, a=b=1.

Compound 13: R1=—H, R2=—CH₂—COO-methyl, R3=—CN, R4=═O, a=1, b=0.

Compound 14: R1=—H, R2=OX1, R3=—CO—X2, R4=NH₂, a=b=1, X1 and X2 eliminated.

Compound 15: R1=—H, R2=COOH, R3=COOH, R4=—NH—CO—NH₂, a=b=1.

Compound 16: R1=—H, R2=OX1, R3=—CO—NHX2, R4=NH₂, a=b=1, X1 and X2 eliminated.

The following counter ions for ionic compounds according to formula I can be used: Na⁺, K⁺, Li⁺, cyclohexylammonium, or basic amino acids (e.g. lysine, arginine, ornithine, glutamine).

The drugs produced with the compounds according to the invention may be administered in an oral, intramuscular, periarticular, intraarticular, intravenous, intraperitoneal, subcutaneous, or rectal manner.

The invention also relates to methods for preparing drugs which are characterized by that at least one compound of formula I is brought into a suitable dosage form by using a pharmaceutically suitable and physiologically well tolerated carrier and if applicable, further suitable active ingredients, or additional or auxiliary substances.

Suitable solid or liquid galenic dosage forms are for instance granulates, powders, dragées, tablets, (micro) capsules, suppositories, syrups, juices, suspensions, emulsions, drops or injectable solutions as well as preparations with protracted release of the active ingredient, prepared according to standard techniques and means such as carrier substances, explosion, binding, coating, swelling, sliding or lubricating agents, flavoring substances, sweeteners and solution mediators are used.

Auxiliary substances are for instance magnesium carbonate, titanium dioxide, lactose, mannite and other sugars, talcum, milk protein, gelatin, starch, cellulose and its derivatives, animal and plant oils such as cod-liver oil, sunflower, peanut or sesame oil, polyethylene glycols and solvents, such as sterile water and one or poly-valent alcohols, e.g. glycerin.

Preferably the drugs are prepared and administered in dosage units, each unit containing as an active component a defined dose of the compound according formula I of the invention. With solid dosage units such as tablets, capsules, dragées or suppositories, this dose may be 1 to 1,000 mg, preferably 50 to 300 mg, and for injection solutions in an ampule form 0.3 to 300 mg, preferably 10 to 100 mg.

For treating an adult patient of 50 to 100 kg weight, for instance 70 kg, for instance daily doses of 20 to 1,000 mg active ingredient, preferably 100 to 500 mg, are indicated. Under certain circumstances, higher or lower daily doses may be recommendable. The administration of the daily dose may be a one-off administration in the form of a single dosage unit or several smaller dosage units as well as a multi-administration of separated doses in certain intervals.

In the following, the invention is explained in more detail with reference to examples representing embodiments only.

### Example 1

The compound carbomethoxypropionyl cyanide was produced according to Q. Tang and S. Sen (Tetrahedron Letters 39 1998, p. 2249-2252). Typically, 1.5 g (10 mmole) carbomethoxypropionyl cyanide were added to a solution of 1.79 g CuCN (20 mmole) in 10 ml acetonitrile. The mixture was heated under reflow for 30 min and concentrated with the Rotavapor after cooling-down to ambient temperature. The residue was dissolved in ether, and the ether solution was filtrated. After removal of the solvent, a slightly yellow oil was left (yield 0.96 g, 67%; IR (cm⁻¹) 2225, 1727.

### Example 2

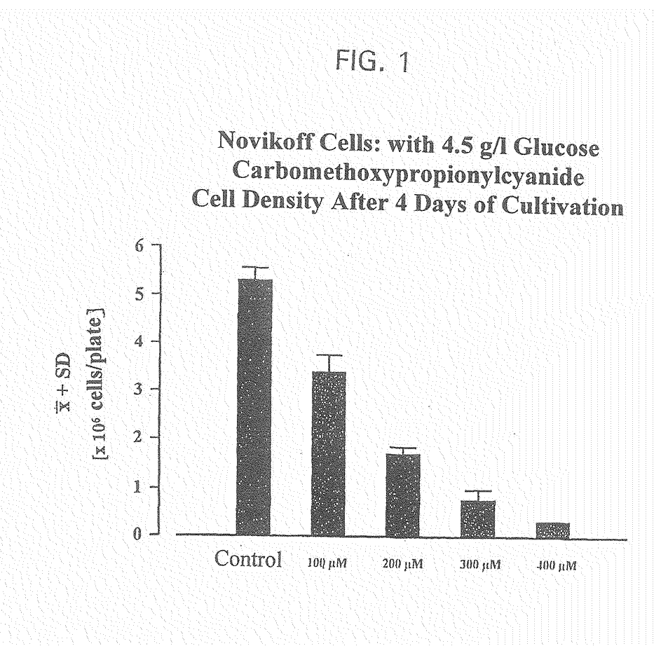

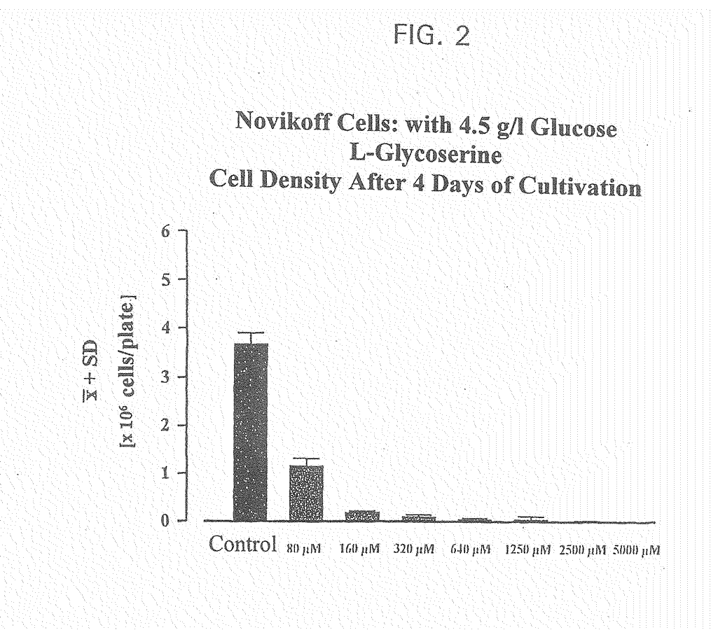

The Novikoff hepatoma cells utilized in this example were obtained from the tumor bank of the Deutsches Krebsforschungszentrum, Heidelberg (Cancer Research 1951, 17, 1010). 100,000 cells each are seeded per 25 cm² cultivation bottle. The substance according to Example 1 of the invention, dissolved in a solvent suitable for use in cell cultures, for instance water, diluted ethanol, dimethylsulfoxide or the like, was added in an increasing concentration to the culture medium, e.g. L-cycloserine (compound 16) or dehydrothreonine (compound 2) in a concentration range of 80 μM-5,000 μM; carbomethoxypropionyl cyanide (compound 13) in a range of 100 μM-300 μM. After four days of cultivation, the number of cells per bottle was counted. The results are shown in FIGS. 1 and 2, and a dose dependence of the proliferation inhibition compared to the control sample without addition of a compound according to the invention can be seen.

### Example 3

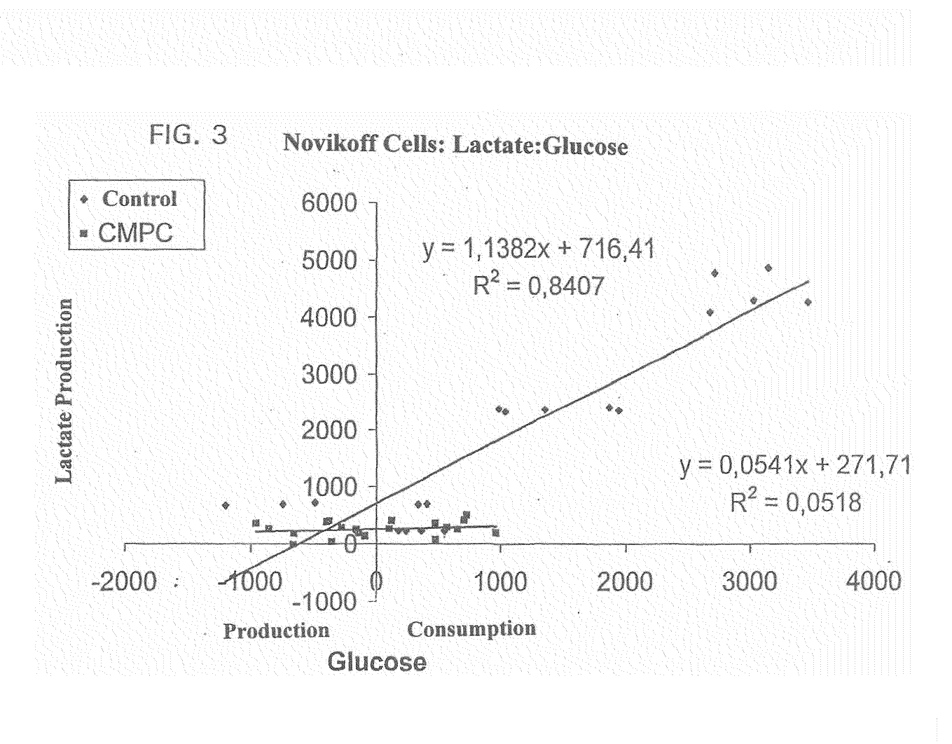

The investigations of carbomethoxypropionyl cyanide (CMPC) for the metabolism of the Novikoff cells showed that CMP massively inhibits the glycolysis flow, as can be seen from FIG. 3.

1. A method of treatment and/or prophylaxis of illnesses from the group consisting of neoplastic tumors, inflammatory diseases, autoimmune diseases, degenerative joint diseases, rheumatic diseases with cartilage breakdown, chronic polyarthritis, joint trauma, immobilization-caused cartilage loss, septic shock, diseases with disturbed leukocyte adhesion, diseases by increased TNFalpha concentrations, cachexia, Crohn's disease, rejection reactions after transplantations, comprising administering a therapeutically effective dose of a pharmaceutical composition according to formula I 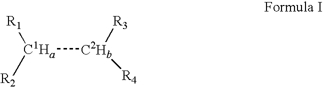
wherein a and b are identical or different and are 0 or 1,
wherein R1=—H, C1-C18 alkyl, cycloalkyl or aryl,
wherein R2=—OX1, —SX1, —COO⁻, —(CH₂)ₙ—COOX1 or —COOX1 with X1=—H, C1-C18 alkyl, cycloalkyl or aryl, and with n=1-8,
wherein R3=—CN, —COO⁻, —COOX2, —C0-X2, —CO—NHX2 with X2=—H, C1-C18 alkyl, cycloalkyl or aryl,
wherein R4=═O, —NHY or —CO—NHZ with Y=H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or C1-C8 alkyl, cycloalkyl or aryl substituted phenyl or with -Hal and/or —O-Hal and/or C1-C8 alkyl, cycloalkyl or aryl substituted naphthyl (-Hal=—F, —Cl, or —Br),
wherein a and b correspond to the number of remaining carbon valences at C¹ and C²,
wherein via R3 a ring connection to C¹ under elimination of X1 in R2 and X2 in R3 may be provided,
or of a physiologically well tolerated salt of such a compound.

2. A compound according to formula I 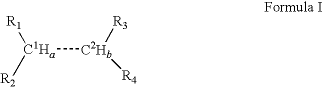
wherein a and b are identical or different and are 0 or 1,
wherein R1=—H, C1-C18 alkyl, cycloalkyl or aryl,
wherein R2=—OX1, —SX1, —COO⁻, —(CH₂)ₙ—COOX1 or —COOX1 with X1=—H, C1-C18 alkyl, cycloalkyl or aryl, and with n=1-8,
wherein R3=—CN,
wherein R4=═O, —NHY or —CO—NHZ with Y=H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or C1-C8 alkyl, cycloalkyl or aryl substituted phenyl or with -Hal and/or —O-Hal and/or C1-C8 alkyl, cycloalkyl or aryl substituted naphthyl (-Hal=—F, —Cl, or —Br),
wherein a and b correspond to the number of remaining carbon valences at C¹ and C², or a physiologically well tolerated salt of such a compound.

3. A pharmaceutical composition containing a compound according to formula I 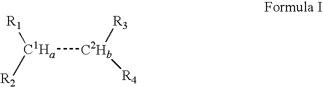
wherein a and b are identical or different and are 0 or 1,
wherein R1=—H, C1-C18 alkyl, cycloalkyl or aryl,
wherein R2=—OX1, —SX1, —COO⁻, —(CH₂)ₙ—COOX1 or —COOX1 with X1=—H, C1-C18 alkyl, cycloalkyl or aryl, and with n=1-8,
wherein R3=—CN, —COO⁻, —COOX2, —C0-X2, —CO—NHX2 with X2=—H, C1-C18 alkyl, cycloalkyl or aryl,
wherein R4=═O, —NHY or —CO—NHZ with Y=H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or C1-C8 alkyl, cycloalkyl or aryl substituted phenyl or with -Hal and/or —O-Hal and/or C1-C8 alkyl, cycloalkyl or aryl substituted naphthyl (-Hal=—F, —Cl, or —Br),
wherein a and b correspond to the number of remaining carbon valences at C¹ and C²,
wherein via R3 a ring connection to C¹ under elimination of X1 in R2 and X2 in R3 may be provided,
or a physiologically well tolerated salt of such a compound, and
at least one physiologically well tolerated auxiliary and/or carrier substance.

4. A pharmaceutical composition according to claim 3, wherein the pharmaceutical composition additionally contains an active ingredient different from the compound of formula I.

5. The method of claim 1, wherein
R1=—H, methyl or ethyl,
R2=—OX, —COO⁻, or —COOX with X=—H, methyl or ethyl, R4=═O, —NHY with Y=H or —COR (R=methyl, ethyl or —NHA with A=H, methyl or ethyl) or —CO—NHZ with Z=—F, —Br, —Cl, —O—Cl, and/or —O—Br substituted phenyl.

6. The method of claim 1, wherein the pharmaceutical composition additionally contains an active ingredient different from the compound of formula I.

7. The compound of claim 2, wherein
R1=—H, methyl or ethyl,
R2=—OX, —COO⁻, or —COOX with X=—H, methyl or ethyl, R4=═O, —NHY with Y=H or —COR(R=methyl, ethyl or —NHA with A=H, methyl or ethyl) or —CO—NHZ with Z=—F, —Br, —Cl, —O—Cl, and/or —O—Br substituted phenyl.

8. The pharmaceutical composition of claim 3 or 4, wherein
R1=—H, methyl or ethyl,
R2=—OX, —COO⁻, or —COOX with X=—H, methyl or ethyl, R4=═O, —NHY with Y=H or —COR (R=methyl, ethyl or —NHA with A=H, methyl or ethyl) or —CO—NHZ with Z=—F, —Br, —Cl, —O—Cl, and/or —O—Br substituted phenyl.
